(19) United States
(12) Patent Application Publication
(10) Pub. No.: US 2004/0096520 A1
(43) Pub. Date: May 20, 2004

(54) Manganese compositions for reducing/preventing skin wrinkles and fine lines

(75) Inventors: Isabelle Nonotte, Paris (FR); Lionel Breton, Versailles (FR)

Correspondence Address: BURNS DOANE SWECKER & MATHIS L L P, POST OFFICE BOX 1404, ALEXANDRIA, VA, 22313-1404, US

(73) Assignee: SOCIETE L'OREAL S.A., Paris (FR)

(21) Appl. No.: 10/702,736

(22) Filed: Nov. 7, 2003

Related U.S. Application Data
(63) Continuation of application No. 09/859,384, filed on May 18, 2001.

(30) Foreign Application Priority Data
May 18, 2000 (FR) 00/06373

(51) Int. Cl. A61K 33/32; A61K 31/28

(52) U.S. Cl. 424/639; 514/492

(57) ABSTRACT

A regime/regimen for relaxing and/or slackening human cutaneous and/or subcutaneous tissue, in particular for treating wrinkles and fine lines in the skin, comprising administering to an individual subject in need of such treatment, a thus-effective amount of manganese and/or at least one organic and/or inorganic salt thereof.

### CROSS-REFERENCE TO PRIOR UNITED STATES APPLICATION

This application is a continuation of copending U.S. application Ser. No. 09/859,384, filed May 18, 2001, incorporated by reference herein in its entirety and relied upon.

### CROSS-REFERENCE TO PRIORITY APPLICATION

This application claims priority under 35 U.S.C. §119 of FR-00/06373, filed May 18, 2000, hereby expressly incorporated by reference.

### CROSS-REFERENCE TO COMPANION APPLICATION

Our copending U.S. application Ser. No. 09/859,384 [Attorney Docket No. 016800-443], filed concurrently with parent application Ser. No. 09/859,384 and assigned to the assignee hereof, and its continuation U.S. application Ser. No. 10/665,372 [Attorney Docket No. 016800-643], filed Sep. 22, 2003.

### BACKGROUND OF THE INVENTION

1. Technical Field of the Invention

The present invention relates to the administration, to individuals in need of such treatment, of cosmetic/dermatological compositions comprising effective amounts of manganese or salts thereof for relaxing and/or slackening cutaneous and/or subcutaneous tissue, notably for reducing/preventing wrinkles and fine lines in the skin.

2. Description of the Prior Art

There is a current trend for women, and even men, to wish to appear young for as long as possible and, consequently, to fade out the signs and marks of aging of the skin, which are especially reflected in wrinkles and fine lines. In this respect, advertising and the fashion world report products intended to maintain a radiant and wrinkle-free skin for as long as possible, which are signs of youthful skin, and all the more so since the physical appearance positively reinforces the psyche and/or morale.

Heretofore, wrinkles and fine lines were treated with cosmetic products containing active agents that act on the skin, for example by moisturizing it or by improving cell renewal, or, alternatively, by promoting the synthesis of collagen, of which skin tissue is composed. However, it was to date unknown to elicit an effect on wrinkles by influencing the contractile elements present in the skin.

Thus, it is known that the facial skin muscles are under the control of motor nerve afferences of the facial nerve and that, moreover, the interlobular partitions of the hypoderm contain within them fibers which constitute a striated muscle tissue (panniculus carnosus). Moreover, it too is known that a subpopulation of dermal fibroblasts, which are known as myofibroblasts, exhibits contractile characteristics in common with muscle tissue.

Calcium is the final messenger of muscle contraction. The contraction/relaxation cycle is due to variations in the cytoplasmic calcium concentration of from 10⁻¹ to 10⁻⁵ M in the contractile cell.

In muscle at rest, the intracellular concentration of free calcium remains less than 10⁻⁸ M, although the extracellular concentration is 10,000 times higher and the force represented by the electrochemical potential gradient tends to influence the calcium to enter the cell. This resting state is due to the low permeability of the cell membrane to calcium and to the activity of various mechanisms which sequester calcium or expel it from the cell. Various cytoplasmic proteins, in particular parvalbumins, thus have the capacity to bind calcium. Among the intracellular organelles, the endoplasmic reticulum can accumulate and release calcium under conditions that are compatible with physiological regulation.

Raising the level of calcium in the muscle cell cytoplasm allows activation of the contractile machinery. The entry of calcium into the intracellular compartment (depolarization) plays a part in reducing the potential difference between the outside and the inside and thus renders the cell more excitable.

Specifically, the depolarization of the transverse tubules (invagination of the cell membrane) which spreads to the longitudinal tubules (sarcoplasmic reticulum) induces the instantaneous release of intracellular calcium by these longitudinal tubules. In the presence of calcium, the contractile proteins of striated muscle show ATPase activity which provides the energy required for the contraction.

Conversely, relaxation of the striated muscle appears following the binding of ATP to the contractile proteins. The intracellular calcium then reenters the intracellular compartment and its concentration returns to a value close to 10⁻⁸ M.

Moreover, it has been shown that botulinum toxin which was originally used to treat spasms, can act on muscular spasticity states (see A. Blitzer et al., Arch. Otolaryngol, Head Neck Surg., 119, pages 1018 to 1022 (1993)) and on wrinkles of the glabella, which are the wrinkles between the eyebrows (see J. D. Carruters et al., J. Dermatol. Surg. Oncol., 18, pages 17 to 21 (1992)). Consequently, it is possible to act on the muscular contractile component of wrinkles (in particular on the motor plate which corresponds to the nerve/muscle junction).

In the peripheral nervous system, the junction between a nerve and a muscle constitutes the neuromuscular plate, upstream of which is the efferent nerve pathway known as the motor neurone. Moreover, the cell membranes of each nerve fiber also comprise many ion channels, and in particular calcium channels, which are capable of allowing the corresponding element to permeate in ionic form, which in this particular case is calcium.

The important role of calcium and of regulating its intracellular concentration in the phenomena of muscle contraction/relaxation, whether these phenomena are pre- or post-nerve cell/non-nerve cell (myocytes, myofibroblasts, etc.) junction, will thus be appreciated.

Regulation of the intracellular calcium concentration is possible only because the efflux of calcium corrects the influx. This can be assured only by an expulsion of the cellular calcium by one or more mechanisms capable of overcoming the electrochemical potential gradient mentioned above.

Two types of mechanism may be involved: a calcium pump which actively expels the cations at the expense of the hydrolysis of ATP and a passive movement of calcium through different channels (dependent on the intracellular and extracellular calcium gradient). In most cells, the ATP-dependent calcium pump operates more efficiently in the presence of calmodulin which increases its affinity.

In order to better describe the calcium-permeability changes, it is currently common to consider that this permeability corresponds to the opening of membrane-bound calcium channels, these channels being operated by variations in the membrane potential (VOC) or by activation of the membrane-bound receptors (ROC). To date, six VOC types of calcium channel (L, N, T, P, Q and R) have been identified.

It will also be appreciated from the foregoing that the contraction or hypercontraction of certain facial muscles results in the appearance of wrinkles. This muscle activation is itself partly induced by a variation in calcium flux through the transmembrane calcium channels.

### SUMMARY OF THE INVENTION

After numerous clinical tests, it has now unexpectedly and surprisingly been determined that contractile muscle fiber, which is under the direct control of the neuromotor influx, serves an essential function in the formation of wrinkles and that the modulation of the neuromotor influx and the control of the contraction of muscle fibers play an essential role in the formation of wrinkles. Thus, it has now been found that the modulation of motor contraction attenuates not only wrinkles but also fine lines and also exerts a “smoothing” effect on the skin's microrelief. It has also now been found that cutaneous and subcutaneous tissues comprise calcium channels, a hitherto unknown phenomenon.

Briefly, the present invention features administering manganese values and influencing the calcium channels of cutaneous and subcutaneous tissues to relax or slacken same and thus reduce wrinkles and fine lines.

### DETAILED DESCRIPTION OF BEST MODE AND SPECIFIC/PREFERRED EMBODIMENTS OF THE INVENTION

More particularly according to the present invention, since 1965, studies have been conducted by T. Godfraind to investigate the mechanisms by which certain medicinal substances inhibit the contractile response to several vasoactive agents. The hypothesis proposed was that the permeability of the membrane to calcium might be inhibited by pharmacological agents, which would constitute the common mechanism on which multi-purpose antagonists would act.

The simplest experimental technique for demonstrating that a pharmacological agent is capable of inhibiting calcium influx entails preincubating a smooth muscle in a calcium-free physiological solution, depolarizing it in a KCl-rich solution and gradually increasing the calcium concentration in the infusion solution. This elicits an increase in the tension of the muscle, the value of which increases to a maximum as a function of the calcium concentration. When this protocol is repeated in the presence of a substance considered to inhibit calcium influx, as was carried out for the first time with cinnarizine, the contractile responses are inhibited in a dose-dependent manner. A similar concept was applied to describe the action of verapamil on the heart. Verapamil was first considered a P-blocker, but its action is more complex since it exerts inhibitory action on excitation/contraction coupling. On the papillary muscle, verapamil suppresses the contraction by very slightly modifying the action potential. It is this observation which resulted in verapamil being considered as a calcium antagonist.

Manganese is a metal which is very widespread at the surface of the earth's crust. It belongs to Group VIIa of the Periodic Table, its atomic number is 25 and its atomic weight is 54.93. Manganese has several valences (1 to 7), the divalent and trivalent forms being those that are the most biologically active.

Manganese is widely used in the metallurgy industry, in the manufacture of dry batteries and as a colorant.

Plants are all rich in Mn, particularly seeds (about 7 μg/g), nuts (about 17 μg/g) and tea. Fruits (about 1 μg/g) and vegetables (about 2.5 μg/g) are less rich, but their level is still very high compared with foods of animal origin (meats: about 0.20 μg/g, fish: about 0.05 μg/g).

Conversely, this metal only exists in trace amounts in animals, particularly humans.

However, its biological role is very important and, even though the harmful effects of a Mn deficiency have not been determined irrefutably in man, the consequences of deficiencies examined in animals indicate that this metal is involved in many metabolic schemes. However, even today, the knowledge regarding the intimate biochemical mechanisms of Mn remains very fragmented.

Manganese has been implicated in many metabolic pathways:

(a) clotting;

(b) thermogenesis (by its action on the thyroid system);

(c) immunity, in which manganese appears to be necessary for a proper synthesis of antibodies;

(d) reproduction, its deficiency, which promotes a reduction in the fertility of females, and of males, may be due to the limiting action of manganese on the synthesis of cholesterol and of sexual hormone precursors.

Two properties permit explaining certain of the physiopathological roles of manganese:

(1) The activation of numerous enzymes:

Manganese is a metal which activates numerous enzymes and lectins. It intervenes either as a dissociable element or by forming an integral part of the structure of the enzyme (metalloenzymes).

(2) Its inhibitory activity on calcium channels.

The internal signal to activate a cell is often triggered by a modification of the intracytoplasmic calcium concentrations. The calcium binds to proteins (calmoduline) which in turn activate kinases. Calcium serves in the transmission of the nerve influx, in the stimulation of certain secretory cells, it triggers the changes in the shape of platelets at the beginning of their activation, etc.

Manganese blocks the penetration of calcium into the cytoplasm in many cells exhibiting secretory activity (for example pancreas), or electric activity; in particular, it inhibits the output of neurotransmitters at the motor plate. Manganese exerts inhibitory action on the stimulation of B and T lymphocytes, if it is added to the medium, a very short time after mitogenesis.

In order for a substance to be recognized as a calcium-channel inhibitor, also referred to herein as a calcium antagonist, it must be able to reduce the intracellular calcium concentration or reduce the binding of calcium to intracellular proteins such as, for example, calmoduline, as is especially described, for example, by Galizzi, J. P. et al., Biol. Chem., 262, p. 6947 (1987) or Y. Okamiya et al., Eur. J. Pharmacol., 205, p. 49 (1991) or J. A. Wagner et al., J. Neurosci., 8, p. 3354 (1988) or H. R. Lee et al., Life Sci., 35, p. 721 (1984) or Schoemaker H. and Lauger S., Eur. J. Pharmacol., 111, p. 273 (1985) or I. J. Reynolds et al., J. Pharmacol. Exp. Ther., 237, p. 731 (1986).

For the purposes of the present invention, a substance is recognized as a relaxer when it elicits a relaxation effect on a contracted muscle tissue and/or exerts an inhibitory effect in an experiment model of nerve/muscle junction (motor plate), in particular, in the model described by W. Steinbrecher in “Electrodes for stimulation and bioelectric potential recording,” Ed. Biomerstechnich, pages 96-98 (1988).

Manganese and the salts thereof fully satisfy these criteria.

As indicated above, the present invention features influencing calcium channels to relax or slacken vascular tissues, and thus combating wrinkles and fine lines, via administering, to an individual subject in need of such treatment, manganese, whether in ionic form, in the form of a salt or in the form of manganese-rich natural, plant or microorganism, particularly bacterial, extracts.

Thus, this invention features the administration of compositions comprising an effective amount of manganese and/or at least one of the salts thereof, to relax and/or slacken cutaneous and/or subcutaneous tissue, such compositions containing a physiologically acceptable medium (vehicle, diluent or carrier).

This invention also features, if necessary formulated into a physiologically acceptable medium therefor, administration of an effective amount of natural, plant or microorganism, particularly bacterial, extracts that are rich in manganese or in manganese salt, to relax and/or slacken cutaneous and/or subcutaneous tissue.

By the term “manganese salts” are intended organic or inorganic manganese salts.

Exemplary organic salts according to the invention include manganese carbonate, manganese acetate, manganese citrate, manganese oleate, manganese oxalate, etc.

And exemplary inorganic manganese salts include the mineral salts, for instance manganese chloride, manganese borate, manganese nitrate, manganese phosphate, manganese sulfate, etc.

Moreover, except where otherwise indicated, the term “manganese” means manganese which is not only in ionic form but also in the form of salts or in the form of manganese-rich natural, plant or microorganism, particularly bacterial, extracts.

By the expression “physiologically acceptable medium” is intended a medium which is compatible with the skin, the scalp and/or mucous membranes.

The present invention also features formulating an effective amount of manganese values into physiologically acceptable media to provide compositions suited for smoothing the skin, and also for attenuating and/or eliminating the microrelief of the skin.

The subject compositions are well suited for curatively and/or preventively combating wrinkles and fine lines in the skin.

The subject compositions are particularly well suited for reducing wrinkles and fine lines in the skin.

More particularly, the relaxation and/or slackening of the cutaneous and/or subcutaneous tissue corresponds to a muscular relaxation or slackening.

Thus, the effective amount of manganese which may be administered according to the invention depends on the desired effect and may vary over a wide range.

To provide an order of magnitude, it is intended, according to the invention, to administer manganese in an amount of from 0.0001% to 10% of the total weight of the composition and preferably in an amount of from 0.001% to 5% of the total weight of the composition.

When, according to the invention, a manganese-rich natural, plant or microorganism, particularly bacterial, extract is administered, one skilled in this art can easily adapt the amount of extract such that, in the final analysis, the manganese is administered in the amounts indicated above.

Exemplary manganese-rich natural extracts according to the invention include the extracts of nut or extracts of tea.

The compositions of the invention are intended for cosmetic or dermatological applications. The compositions of the invention are preferably suited for cosmetic applications.

The regime/regimen according to the invention is cosmetic, since intended to modify a person's appearance.

The compositions according to the invention may be in any presentation form that is conventional for topical, injectable or oral administration.

The compositions according to the invention may be administered either locally, i.e., topically, or by subcutaneous and/or intradermal injection or by oral administration.

Preferably, the subject compositions are topically applied.

The amounts of the various constituents of the compositions according to the invention are those conventionally included in the fields under consideration and which are appropriate for the various formulations.

For topical application, the compositions of the invention comprise a medium that is compatible with the skin. These compositions may especially be formulated as aqueous, alcoholic or aqueous/alcoholic solutions, gels, water-in-oil or oil-in-water emulsions having the appearance of a cream or a gel, microemulsions or aerosols, or alternatively in the form of vesicular dispersions containing ionic and/or nonionic lipids. Such compositions are formulated according to the usual methods in the fields under consideration.

These topical-application compositions may, in particular, constitute a protective or care formulation for the face, the neck, the hands or the body (for example day creams, night creams, sunscreen creams or oils or body milks), a makeup composition (for example a foundation) or an artificial tanning composition.

When the composition of the invention is an emulsion, the proportion of fatty substance it contains may range from 5% to 80% by weight and preferably from 5% to 50% by weight relative to the total weight of the composition. The fatty substances and emulsifiers contained in the emulsion are selected from among those conventionally employed in the cosmetic or pharmaceutical field.

Exemplary fatty substances according to the invention include the mineral oils (petroleum jelly), plant oils (liquid fraction of karite butter) and hydrogenated derivatives thereof, animal oils, synthetic oils (perhydrosqualene), silicone oils (polydimethylsiloxane) and fluoro oils. Other fatty substances which are representative are the fatty alcohols (cetyl alcohol and stearyl alcohol), fatty acids (stearic acid) and waxes.

The emulsifiers are advantageously present in the compositions in a proportion ranging from 0.3% to 30% by weight and preferably from 0.5% to 30% by weight relative to the total weight of the composition.

In known fashion, the compositions of this invention may also contain adjuvants and additives that are common in the corresponding fields, such as hydrophilic or lipophilic gelling agents, preservatives, antioxidants, solvents, fragrances, fillers, UV-screening agents, dyestuffs, colorants, etc. Moreover, the subject compositions may contain hydrophilic or lipophilic bioaffecting active agents. The amounts of these various adjuvants, additives or active agents are those that are conventional in the cosmetic or pharmaceutical field, and, for example, range from 0.01% to 20% of the total weight of the composition. Depending on their nature, these adjuvants, additives or active agents may be introduced into the fatty phase, into the aqueous phase and/or into lipid vesicles.

Among the active agents which the compositions of the invention may contain, particularly exemplary are the active agents which have an effect on the treatment of wrinkles or fine lines, other than manganese, and in particular keratolytic active agents. By the term “keratolytic” is intended an active agent which has desquamating, exfoliant or scrubbing properties, or an active agent capable of softening the horny layer.

Exemplary active agents for the treatment of wrinkles or fine lines, which the compositions of the invention may contain, include alverine or salts thereof, chlorine-channel openers, hydroxy acids and retinoids.

The hydroxy acids may be, for example, a-hydroxy acids or P-hydroxy acids, which may be linear, branched or cyclic, and saturated or unsaturated. The hydrogen atoms of the carbon chain may also be substituted with halogens, or halogenated alkyl, acyl, acyloxy, alkoxycarbonyl or alkoxy radicals having from 2 to 18 carbon atoms.

Exemplary hydroxy acids include, in particular, glycolic acid, lactic acid, maleic acid, tartaric acid, citric acid, 2-hydroxyalkanoic acid, mandelic acid, salicylic acid and alkyl derivatives thereof, for instance 5-n-octanoylsalicylic acid, 5-n-dodecanoylsalicylic acid, 5-n-decanoylsalicylic acid, 5-n-octylsalicylic acid, 5-n-heptyloxysalicylic acid or 4-n-heptyloxysalicylic acid and 2-hydroxy-3-methylbenzoic acid, or alkoxy derivatives thereof, for instance 2-hydroxy-3-methoxybenzoic acid.

And exemplary retinoids include, in particular, retinoic acid (all-trans or 13-cis) and derivatives thereof, retinol (vitamin A) and esters thereof, such as retinyl palmitate, retinyl acetate and retinyl propionate, and the salts thereof.

These active agents may be formulated, in particular, at concentrations ranging from 0.0001% to 5% by weight relative to the total weight of the composition.

The present invention also features a cosmetic regime/regimen for treating wrinkles and/or fine lines, comprising topically applying onto the skin a cosmetic composition containing an effective amount of manganese, formulated into a physiologically acceptable medium.

The cosmetic regime/regimen of the invention may be carried out, in particular, by topically applying the cosmetic composition as described above, via usual techniques. For example: application of creams, gels, sera, lotions, makeup-removing milks or sunscreen compositions onto the skin or application of spray compositions.

In order to further illustrate the present invention and the advantages thereof, the following specific examples are given, it being understood that same are intended only as illustrative and in nowise limitative.

In said examples to follow, all parts and percentages are given by weight, unless otherwise indicated.

### EXAMPLE 1

Activity of manganese gluconate and of manganese chloride in a model of nerve/muscle junction (motor plate) obtained in a phrenic nerve/isolated diaphragm preparation: investigation of a myorelaxant effect:

The phrenic nerve and the diaphragm were carefully isolated and placed in a 50 ml tank filled with survival fluid (Krebs Henseleit liquid) maintained at a temperature of 37° C. and oxygenated with a mixture of 95% oxygen and 5% CO₂.

The variations in the tension of the diaphragm were then recorded with an initial preloading of several grams.

After a relaxation period of 30 min., the diaphragm was stimulated indirectly via the phrenic nerve.

On each preparation, the effect of the test products was evaluated, in a first stage, on the contractions induced by indirect stimulation via stimulation of the phrenic nerve (0.1 to 7 volts, 0.3 ms, 0.1 Hz) at increasing and cumulative concentrations from 10⁻⁶ M to 10⁻³ M.

The results obtained in the model of motor plate using the 2 manganese salts were as reported in the following Table:

TABLE

| | | % of inhibition (indirect |
|---|---|---|
| Product | Concentration | stimulation) |
| Manganese gluconate (n = 2) | 10⁻³ M | 25% |
| MnCl₂ | 10⁻³ M | 10% |

### EXAMPLES 2-6

The following are specific examples of formulations according to the invention:

### EXAMPLE 2

Composition 1: Anti-wrinkle care lotion for the face:

| | |
|---|---|
| Manganese gluconate | 1.50% |
| Antioxidant | 0.05% |
| Preservative | 0.30% |
| Ethanol (solvent) | 8.00% |
| Water | qs 100% |

This lotion acts on wrinkles during repeated use (application twice daily for one month).

### EXAMPLE 3

Composition 2: Care gel for the face:

| | | |
|---|---|---|
| Manganese chloride | | 0.50% |
| Hydroxylpropylcellulose* | | 1.00% |
| Preservative | | 0.30% |
| Ethanol (solvent) | | 15.00% |
| Antioxidant | | 0.05% |
| Water | qs | 100% |

*Klucel H marketed by Hercules (gelling agent)

The gel obtained acts on wrinkles. It may be applied daily, morning and evening for one month.

### EXAMPLE 4

Composition 3: Care cream for the face (oil-in-water emulsion):

| | |
|---|---|
| Manganese gluconate | 0.50% |
| Glyceryl stearate (emulsifier) | 2.00% |
| Polysorbate-60 (Tween 60 marketed by | 1.00% |
| ICI) (emulsifier) | |
| Stearic acid | 1.40% |
| Triethanolamine (neutralizer) | 0.70% |
| Carbomer (Carbopol 940 marketed by | 0.40% |
| Goodrich) | |
| Liquid fraction of karite butter | 12.00% |
| Perhydrosqualene | 12.00% |
| Preservative | 0.30% |
| Fragrance | 0.50% |
| Antioxidant | 0.05% |
| Water | qs 100% |

A rich white cream is obtained, which reduces wrinkles and fine lines, and which may be applied daily.

### EXAMPLE 5

Composition 4: Care cream for the face (oil-in-water emulsion):

| | |
|---|---|
| Manganese gluconate | 0.10% |
| Glyceryl monostearate/distearate | 2.00% |
| Cetyl alcohol | 1.50% |
| Mixture of cetylstearyl alcohol/33 EO | 7.00% |
| oxyethylenated cetylstearyl alcohol | |
| Polydimethylsiloxane | 1.50% |
| Liquid petroleum jelly | 17.50% |
| Preservative | 0.30% |
| Fragrance | 0.50% |
| Glycerol | 12.50% |
| Water | qs 100% |

### EXAMPLE 6

Composition 5: Care cream for the face (oil-in-water emulsion):

| | |
|---|---|
| Extract of walnut | 5.00% |
| Glyceryl monostearate/distearate | 2.00% |
| Cetyl alcohol | 1.50% |
| Mixture of cetylstearyl alcohol/33 EO | 7.00% |
| oxyethylenated cetylstearyl alcohol | |
| Polydimethylsiloxane | 1.50% |
| Liquid petroleum jelly | 17.50% |
| Preservative | 0.30% |
| Fragrance | 0.50% |
| Glycerol | 12.50% |
| Water | qs 100% |

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime/regimen for influencing calcium channels in contractile fibers to relax and/or slacken cutaneous and/or subcutaneous tissue, comprising administering to an individual subject in need of such treatment a cosmetic/dermatological composition comprising: manganese, in the form of at least one organic or inorganic manganese salt, in an amount effective to influence calcium channels in contractile fibers to relax and/or slacken cutaneous and/or subcutaneous tissue, said amount being from 0.001% to 0.5% by weight of the composition; and a physiologically acceptable vehicle, diluent or carrier therefor.

2. A regime/regimen according to claim 1 for influencing calcium channels in contractile fibers to relax and/or slacken skin tissue and thus treat wrinkles and fine lines in the skin, comprising topically administering to an individual subject in need of such treatment a cosmetic/dermatological composition comprising: manganese, in the form of at least one organic or inorganic manganese salt, in an amount effective to influence calcium channels in contractile fibers to relax and/or slacken skin tissue and thus treat wrinkles and fine lines, said amount being from 0.001% to 0.5% by weight of the composition; and a topically applicable, physiologically acceptable vehicle, diluent or carrier therefor.

3. A regime/regimen according to claim 1 for influencing calcium channels in contractile fibers to relax and/or slacken cutaneous and/or subcutaneous tissue and thus smooth the skin, comprising topically administering to an individual subject in need of such treatment a cosmetic/dermatological composition comprising: manganese, in the form of at least one organic or inorganic manganese salt, in an amount effective to influence calcium channels in contractile fibers to relax and/or slacken cutaneous and/or subcutaneous tissue and thus smooth the skin, said amount being from 0.001% to 0.5% by weight of the composition; and a topically applicable, physiologically acceptable vehicle, diluent or carrier therefor.

4. A regime/regimen according to claim 1 for influencing calcium channels in contractile fibers to relax and/or slacken cutaneous and/or subcutaneous tissue and thus attenuate and/or eliminate microrelief in the skin, comprising topically administering to an individual subject in need of such treatment a cosmetic/dermatological composition comprising: manganese, in the form of at least one organic or inorganic manganese salt, in an amount effective to influence calcium channels in contractile fibers to relax and/or slacken cutaneous and/or subcutaneous tissue and thus attenuate and/or eliminate microrelief in the skin, said amount being from 0.001% to 0.5% by weight of the composition; and a topically applicable, physiologically acceptable vehicle, diluent or carrier therefor.

5. A regime/regimen according to claim 1 for influencing calcium channels in contractile fibers to relax and/or slacken cutaneous and/or subcutaneous tissue and to thus reduce wrinkles and/or fine lines in the skin, and/or to thus smooth the skin and/or to thus attenuate and/or eliminate microrelief in the skin, comprising topically administering to an individual subject in need of such treatment a cosmetic/dermatological composition comprising: manganese, in the form of at least one organic or inorganic manganese salt, in an amount effective to influence calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, said amount being from 0.001% to 0.5% by weight of the composition; and a topically applicable, physiologically acceptable vehicle, diluent or carrier therefor.

6. A regime/regimen for influencing calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, comprising topically administering to an individual subject in need of such treatment a cosmetic/dermatological composition comprising: (i) manganese, in the form of at least one organic or inorganic manganese salt, in an amount effective to influence calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, said amount being from 0.001% to 0.5% by weight of the composition; and (ii) a bioaffecting amount of alverine or salt thereof, a keratolytic agent, a chlorine-channel opener, a hydroxy acid and/or a retinoid; formulated into a topically applicable, physiologically acceptable vehicle, diluent or carrier therefor.

7. A regime/regimen according to claim 6 for influencing calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, comprising topically administering to an individual subject in need of such treatment a cosmetic/dermatological composition comprising: (i) manganese, in the form of at least one organic or inorganic manganese salt, in an amount effective to influence calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, said amount being from 0.001% to 0.5% by weight of the composition; and (ii) a bioaffecting amount of alverine or salt thereof and/or a chlorine-channel opener; formulated into a topically applicable, physiologically acceptable vehicle, diluent or carrier therefor.

8. A regime/regimen according to claim 1, wherein manganese is administered as at least one of manganese gluconate, manganese carbonate, manganese acetate, manganese citrate, manganese oleate, manganese oxalate, manganese chloride, manganese borate, manganese nitrate, manganese phosphate and/or manganese sulfate.

9. The regime/regimen according to claim 1, wherein said composition is administered topically.

10. The regime/regimen according to claim 1, wherein said composition is administered orally.

11. The regime/regimen according to claim 1, wherein said composition is administered by injection.

12. The regime/regimen according to claim 8, wherein said composition is administered topically.

13. The regime/regimen according to claim 9, wherein said cosmetic/dermatological composition is a topically applicable solution, gel, emulsion, cream, oil, milk, makeup, serum, aerosol, vesicular dispersion, or artificial tanning preparation.

14. The regime/regimen according to claim 5, wherein manganese is administered as at least one of manganese gluconate, manganese carbonate, manganese acetate, manganese citrate, manganese oleate, manganese oxalate, manganese chloride, manganese borate, manganese nitrate, manganese phosphate, and/or manganese sulfate.

15. The regime/regimen according to claim 5, wherein said cosmetic/dermatological composition is a topically applicable solution, gel, emulsion, cream, oil, milk, makeup, serum, aerosol, vesicular dispersion, or artificial tanning preparation.

16. The regime/regimen according to claim 6, wherein manganese is administered as at least one of manganese gluconate, manganese carbonate, manganese acetate, manganese citrate, manganese oleate, manganese oxalate, manganese chloride, manganese borate, manganese nitrate, manganese phosphate, and/or manganese sulfate.

17. The regime/regimen according to claim 6, wherein said cosmetic/dermatological composition is a topically applicable solution, gel, emulsion, cream, oil, milk, makeup, serum, aerosol, vesicular dispersion, or artificial tanning preparation.

18. The regime/regimen according to claim 7, wherein manganese is administered as at least one of manganese gluconate, manganese carbonate, manganese acetate, manganese citrate, manganese oleate, manganese oxalate, manganese chloride, manganese borate, manganese nitrate, manganese phosphate, and/or manganese sulfate.

19. The regime/regimen according to claim 7, wherein said cosmetic/dermatological composition is a topically applicable solution, gel, emulsion, cream, oil, milk, makeup, serum, aerosol, vesicular dispersion, or artificial tanning preparation.

20. A topically applicable cosmetic/dermatological composition suited for influencing calcium channels in contractile fibers to relax and/or slacken skin tissue, said composition comprising: (i) manganese, in the form of at least one organic or inorganic manganese salt, in an amount effective to influence calcium channels in contractile fibers to relax and/or slacken skin tissue, said amount being from 0.001% to 0.5% by weight of the composition; and (ii) a bioaffecting amount of alverine or salt thereof and/or a chlorine-channel opener; formulated into a topically applicable, physiologically acceptable vehicle, diluent or carrier therefor.

21. The cosmetic/dermatological composition according to claim 20, wherein the manganese is present as at least one of manganese gluconate, manganese carbonate, manganese acetate, manganese citrate, manganese oleate, manganese oxalate, manganese chloride, manganese borate, manganese nitrate, manganese phosphate, and/or manganese sulfate.

22. The cosmetic/dermatological composition according to claim 20, said composition being in the form of a topically applicable solution, gel, emulsion, cream, oil, milk, makeup, serum, aerosol, vesicular dispersion, or artificial tanning preparation.

23. A regime/regimen for influencing calcium channels in contractile fibers to relax and/or slacken cutaneous and/or subcutaneous tissue, comprising topically administering to an individual subject in need of such treatment a cosmetic/dermatological composition consisting of: manganese, in the form of at least one organic or inorganic manganese salt, in an amount effective to influence calcium channels in contractile fibers to relax and/or slacken cutaneous and/or subcutaneous tissue; water; and at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants, keratolytic agents, alverine, alverine salts, chlorine-channel openers, hydroxy acids and retinoids.

24. A regime/regimen for influencing calcium channels in contractile fibers to relax and/or slacken cutaneous and/or subcutaneous tissue, comprising topically administering to an individual subject in need of such treatment a cosmetic/dermatological composition consisting of: manganese, in the form of at least one organic or inorganic manganese salt, in an amount effective to influence calcium channels in contractile fibers to relax and/or slacken cutaneous and/or subcutaneous tissue; water; and at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants, anti-wrinkle agents, keratolytic agents, alverine, alverine salts, chlorine-channel openers, hydroxy acids and retinoids.

25. A regime/regimen according to claim 23 for influencing calcium channels in contractile fibers to relax and/or slacken cutaneous and/or subcutaneous tissue, comprising topically administering to an individual subject in need of such treatment a cosmetic/dermatological composition consisting of: manganese, in the form of at least one organic or inorganic manganese salt, in an amount effective to influence calcium channels in contractile fibers to relax and/or slacken cutaneous and/or subcutaneous tissue, said amount being from 0.001% to 0.5% by weight of the composition; water; and at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants, keratolytic agents, alverine, alverine salts, chlorine-channel openers, hydroxy acids and retinoids.

26. A regime/regimen according to claim 24 for influencing calcium channels in contractile fibers to relax and/or slacken cutaneous and/or subcutaneous tissue, comprising topically administering to an individual subject in need of such treatment a cosmetic/dermatological composition consisting of: manganese, in the form of at least one organic or inorganic manganese salt, in an amount effective to influence calcium channels in contractile fibers to relax and/or slacken cutaneous and/or subcutaneous tissue, said amount being from 0.001% to 0.5% by weight of the composition; water; and at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants, anti-wrinkle agents, keratolytic agents, alverine, alverine salts, chlorine-channel openers, hydroxy acids and retinoids.

27. A regime/regimen for influencing calcium channels in contractile fibers to relax and/or slacken cutaneous and/or subcutaneous tissue, comprising topically administering to an individual subject in need of such treatment a cosmetic/dermatological composition consisting of: at least one form of manganese selected from the group consisting of organic salts of manganese, inorganic salts of manganese, manganese-rich plant extracts and manganese-rich microorganism extracts, in an amount effective to influence calcium channels in contractile fibers to relax and/or slacken cutaneous and/or subcutaneous tissue; water; and at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants, kerotolytic agents, alverine, alverine salts, chlorine-channels openers, hydroxy acids and retinoids.

28. A regime/regimen for influencing calcium channels in contractile fibers to relax and/or slacken cutaneous and/or subcutaneous tissue, comprising topically administering to an individual subject in need of such treatment a cosmetic/dermatological composition consisting of: at least one form of manganese selected from the group consisting of organic salts of manganese, inorganic salts of manganese, manganese-rich plant extracts and manganese-rich microorganism extracts, in an amount effective to influence calcium channels in contractile fibers to relax and/or slacken cutaneous and/or subcutaneous tissue; water; and at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants, anti-wrinkle agents, kerotolytic agents, alverine, alverine salts, chlorine-channel openers, hydroxy acids and retinoids.

29. A regime/regimen according to claim 23 for influencing calcium channels in contractile fibers to relax and/or slacken skin tissue and thus treat wrinkles and fine lines in the skin, comprising topically administering to an individual subject in need of such treatment a cosmetic/dermatological composition consisting of: manganese, in the form of at least one organic or inorganic manganese salt, in an amount effective to influence calcium channels in contractile fibers to relax and/or slacken skin tissue and thus treat wrinkles and fine lines; water; and at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants, keratolytic agents, alverine, alverine salts, chlorine-channel openers, hydroxy acids and retinoids.

30. A regime/regimen according to claim 24 for influencing calcium channels in contractile fibers to relax and/or slacken skin tissue and thus treat wrinkles and fine lines in the skin, comprising topically administering to an individual subject in need of such treatment a cosmetic/dermatological composition consisting of: manganese, in the form of at least one organic or inorganic manganese salt, in an amount effective to influence calcium channels in contractile fibers to relax and/or slacken skin tissue and thus treat wrinkles and fine lines; water; and at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants, anti-wrinkle agents, keratolytic agents, alverine, alverine salts, chlorine-channel openers, hydroxy acids and retinoids.

31. A regime/regimen according to claim 23 for influencing calcium channels in contractile fibers to relax and/or slacken cutaneous and/or subcutaneous tissue and thus smooth the skin, comprising topically administering to an individual subject in need of such treatment a cosmetic/dermatological composition consisting of: manganese, in the form of at least one organic or inorganic manganese salt, in an amount effective to influence calcium channels in contractile fibers to relax and/or slacken cutaneous and/or subcutaneous tissue and thus smooth the skin; water; and at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants, keratolytic agents, alverine, alverine salts, chlorine-channel openers, hydroxy acids and retinoids.

32. A regime/regimen according to claim 24 for influencing calcium channels in contractile fibers to relax and/or slacken cutaneous and/or subcutaneous tissue and thus smooth the skin, comprising topically administering to an individual subject in need of such treatment a cosmetic/dermatological composition consisting of: manganese, in the form of at least one organic or inorganic manganese salt, in an amount effective to influence calcium channels in contractile fibers to relax and/or slacken cutaneous and/or subcutaneous tissue and thus smooth the skin; water; and at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants, anti-wrinkle agents, keratolytic agents, alverine, alverine salts, chlorine-channel openers, hydroxy acids and retinoids.

33. A regime/regimen according to claim 23 for influencing calcium channels in contractile fibers to relax and/or slacken cutaneous and/or subcutaneous tissue and thus attenuate and/or eliminate microrelief in the skin, comprising topically administering to an individual subject in need of such treatment a cosmetic/dermatological composition consisting of: manganese, in the form of at least one organic or inorganic manganese salt, in an amount effective to influence calcium channels in contractile fibers to relax and/or slacken cutaneous and/or subcutaneous tissue and thus attenuate and/or eliminate microrelief in the skin; water; and at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants, keratolytic agents, alverine, alverine salts, chlorine-channel openers, hydroxy acids and retinoids.

34. A regime/regimen according to claim 24 for influencing calcium channels in contractile fibers to relax and/or slacken cutaneous and/or subcutaneous tissue and thus attenuate and/or eliminate microrelief in the skin, comprising topically administering to an individual subject in need of such treatment a cosmetic/dermatological composition consisting of: manganese, in the form of at least one organic or inorganic manganese salt, in an amount effective to influence calcium channels in contractile fibers to relax and/or slacken cutaneous and/or subcutaneous tissue and thus attenuate and/or eliminate microrelief in the skin; water; and at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants, anti-wrinkle agents, keratolytic agents, alverine, alverine salts, chlorine-channel openers, hydroxy acids and retinoids.

35. A regime/regimen according to claim 23 for influencing calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, comprising topically administering to an individual subject in need of such treatment a cosmetic/dermatological composition consisting of: manganese, in the form of at least one organic or inorganic manganese salt, in an amount effective to influence calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin; water; and at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants, keratolytic agents, alverine, alverine salts, chlorine-channel openers, hydroxy acids and retinoids.

36. A regime/regimen according to claim 24 for influencing calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, comprising topically administering to an individual subject in need of such treatment a cosmetic/dermatological composition consisting of: manganese, in the form of at least one organic or inorganic manganese salt, in an amount effective to influence calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin; water; and at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants, anti-wrinkle agents, keratolytic agents, alverine, alverine salts, chlorine-channel openers, hydroxy acids and retinoids.

37. A regime/regimen according to claim 25 for influencing calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, comprising topically administering to an individual subject in need of such treatment a cosmetic/dermatological composition consisting of: manganese, in the form of at least one organic or inorganic manganese salt, in an amount effective to influence calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, said amount being from 0.001% to 0.5% by weight of the composition; water; and at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants, keratolytic agents, alverine, alverine salts, chlorine-channel openers, hydroxy acids and retinoids.

38. A regime/regimen according to claim. 26 for influencing calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, comprising topically administering to an individual subject in need of such treatment a cosmetic/dermatological composition consisting of: manganese, in the form of at least one organic or inorganic manganese salt, in an amount effective to influence calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, said amount being from 0.001% to 0.5% by weight of the composition; water; and at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants, anti-wrinkle agents, keratolytic agents, alverine, alverine salts, chlorine-channel openers, hydroxy acids and retinoids.

39. A regime/regimen according to claim 27 for influencing calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, comprising topically administering to an individual subject in need of such treatment a cosmetic/dermatological composition consisting of: at least one form of manganese selected from the group consisting of organic salts of manganese, inorganic salts of manganese, manganese-rich plant extracts and manganese-rich microorganism extracts, in an amount effective to influence calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin; water; and at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants, keratolytic agents, alverine, alverine salts, chlorine-channel openers, hydroxy acids and retinoids.

40. A regime/regimen according to claim 28 for influencing calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, comprising topically administering to an individual subject in need of such treatment a cosmetic/dermatological composition consisting of: at least one form of manganese selected from the group consisting of organic salts of manganese, inorganic salts of manganese, manganese-rich plant extracts and manganese-rich microorganism extracts, in an amount effective to influence calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin; water; and at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants, anti-wrinkle agents, keratolytic agents, alverine, alverine salts, chlorine-channel openers, hydroxy acids and retinoids.

41. A regime/regimen for influencing calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, comprising topically administering to an individual subject in need of such treatment a cosmetic/dermatological composition consisting of: (i) manganese, in the form of at least one organic or inorganic manganese salt, in an amount effective to influence calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin; (ii) a bioaffecting amount of alverine or salt thereof, a keratolytic agent, a chlorine-channel opener, a hydroxy acid and/or a retinoid; (iii) water; and (iv) at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs and colorants.

42. A regime/regimen for influencing calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, comprising topically administering to an individual subject in need of such treatment a cosmetic/dermatological composition consisting of: (i) manganese, in the form of at least one organic or inorganic manganese salt, in an amount effective to influence calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin; (ii) a bioaffecting amount of alverine or salt thereof, a keratolytic agent, a chlorine-channel opener, a hydroxy acid and/or a retinoid; (iii) water; and (iv) at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants and anti-wrinkle agents.

43. A regime/regimen according to claim 41 for influencing calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, comprising topically administering to an individual subject in need of such treatment a cosmetic/dermatological composition consisting of: (i) manganese, in the form of at least one organic or inorganic manganese salt, in an amount effective to influence calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, said amount being from 0.001% to 0.5% by weight of the composition; (ii) a bioaffecting amount of alverine or salt thereof, a keratolytic agent, a chlorine-channel opener, a hydroxy acid and/or a retinoid; (iii) water; and (iv) at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs and colorants.

44. A regime/regimen according to claim 42 for influencing calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, comprising topically administering to an individual subject in need of such treatment a cosmetic/dermatological composition consisting of: (i) manganese, in the form of at least one organic or inorganic manganese salt, in an amount effective to influence calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, said amount being from 0.001% to 0.5% by weight of the composition; (ii) a bioaffecting amount of alverine or salt thereof, a keratolytic agent, a chlorine-channel opener, a hydroxy acid and/or a retinoid; (iii) water; and (iv) at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants and anti-wrinkle agents.

45. A regime/regimen for influencing calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, comprising topically administering to an individual subject in need of such treatment a cosmetic/dermatological composition consisting of: (i) at least one form of manganese selected from the group consisting of organic salts of manganese, inorganic salts of manganese, manganese-rich plant extracts and manganese-rich microorganism extracts, in an amount effective to influence calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin; (ii) a bioaffecting amount of alverine or salt thereof, a keratolytic agent, a chlorine-channel opener, a hydroxy acid and/or a retinoid; (iii) water; and (iv) at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs and colorants.

46. A regime/regimen for influencing calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, comprising topically administering to an individual subject in need of such treatment a cosmetic/dermatological composition consisting of: (i) at least one form of manganese selected from the group consisting of organic salts of manganese, inorganic salts of manganese, manganese-rich plant extracts and manganese-rich microorganism extracts, in an amount effective to influence calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin; (ii) a bioaffecting amount of alverine or salt thereof, a keratolytic agent, a chlorine-channel opener, a hydroxy acid and/or a retinoid; (iii) water; and (iv) at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants and anti-wrinkle agents.

47. A regime/regimen according to claim 41 for influencing calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, comprising topically administering to an individual subject in need of such treatment a cosmetic/dermatological composition consisting of: (i) manganese, in the form of at least one organic or inorganic manganese salt, in an amount effective to influence calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin; (ii) a bioaffecting amount of alverine or salt thereof and/or a chlorine-channel opener; (iii) water; and (iv) at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs and colorants.

48. A regime/regimen according to claim 42 for influencing calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, comprising topically administering to an individual subject in need of such treatment a cosmetic/dermatological composition consisting of: (i) manganese, in the form of at least one organic or inorganic manganese salt, in an amount effective to influence calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin; (ii) a bioaffecting amount of alverine or salt thereof and/or a chlorine-channel opener; (iii) water; and (iv) at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants and anti-wrinkle agents.

49. A regime/regimen according to claim 43 for influencing calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, comprising topically administering to an individual subject in need of such treatment a cosmetic/dermatological composition consisting of: (i) manganese, in the form of at least one organic or inorganic manganese salt, in an amount effective to influence calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, said amount being from 0.001% to 0.5% by weight of the composition; (ii) a bioaffecting amount of alverine or salt thereof and/or a chlorine-channel opener; (iii) water; and (iv) at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs and colorants.

50. A regime/regimen according to claim 44 for influencing calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, comprising topically administering to an individual subject in need of such treatment a cosmetic/dermatological composition consisting of: (i) manganese, in the form of at least one organic or inorganic manganese salt, in an amount effective to influence calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, said amount being from 0.001% to 0.5% by weight of the composition; (ii) a bioaffecting amount of alverine or salt thereof and/or a chlorine-channel opener; (iii) water; and (iv) at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants and anti-wrinkle agents.

51. A regime/regiment according to claim 45 for influencing calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, comprising topically administering to an individual subject in need of such treatment a cosmetic/dermatological composition consisting of: (i) at least one form of manganese selected from the group consisting of organic salts of manganese, inorganic salts of manganese, manganese-rich plant extracts and manganese-rich microorganism extracts, in an amount effective to influence calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin; (ii) a bioaffecting amount of alverine or salt thereof and/or a chlorine-channel opener; (iii) water; and (iv) at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs and colorants.

52. A regime/regiment according to claim 46 for influencing calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin, comprising topically administering to an individual subject in need of such treatment a cosmetic/dermatological composition consisting of: (i) at least one form of manganese selected from the group consisting of organic salts of manganese, inorganic salts of manganese, manganese-rich plant extracts and manganese-rich microorganism extracts, in an amount effective to influence calcium channels in contractile fibers to relax and/or slacken cutaneous and/or subcutaneous tissue and to thus reduce wrinkles and/or fine lines in the skin, and/or to thus smooth the skin, and/or to thus attenuate and/or eliminate microrelief in the skin; (ii) a bioaffecting amount of alverine or salt thereof and/or a chlorine-channel opener; (iii) water; and (iv) at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs, colorants and anti-wrinkle agents.

53. The regime/regimen according to claim 23, wherein said cosmetic/dermatological composition is a topically applicable solution, gel, emulsion, cream, oil, milk, makeup, serum, aerosol, vesicular dispersion, or artificial tanning preparation.

54. The regime/regimen according to claim 24, wherein said cosmetic/dermatological composition is a topically applicable solution, gel, emulsion, cream, oil, milk, makeup, serum, aerosol, vesicular dispersion, or artificial tanning preparation.

55. The regime/regimen according to claim 23, wherein manganese is administered as at least one of manganese gluconate, manganese carbonate, manganese acetate, manganese citrate, manganese oleate, manganese oxalate, manganese chloride, manganese borate, manganese nitrate, manganese phosphate, and/or manganese sulfate.

56. The regime/regimen according to claim 24, wherein manganese is administered as at least one of manganese gluconate, manganese carbonate, manganese acetate, manganese citrate, manganese oleate, manganese oxalate, manganese chloride, manganese borate, manganese nitrate, manganese phosphate, and/or manganese sulfate.

57. The regime/regimen according to claim 25, wherein manganese is administered as at least one of manganese gluconate, manganese carbonate, manganese acetate, manganese citrate, manganese oleate, manganese oxalate, manganese chloride, manganese borate, manganese nitrate, manganese phosphate, and/or manganese sulfate.

58. The regime/regimen according to claim 26, wherein manganese is administered as at least one of manganese gluconate, manganese carbonate, manganese acetate, manganese citrate, manganese oleate, manganese oxalate, manganese chloride, manganese borate, manganese nitrate, manganese phosphate, and/or manganese sulfate.

59. The regime/regimen according to claim 41, wherein said cosmetic/dermatological composition is a topically applicable solution, gel, emulsion, cream, oil, milk, makeup, serum, aerosol, vesicular dispersion, or artificial tanning preparation.

60. The regime/regimen according to claim 42, wherein said cosmetic/dermatological composition is a topically applicable solution, gel, emulsion, cream, oil, milk, makeup, serum, aerosol, vesicular dispersion, or artificial tanning preparation.

61. The regime/regimen according to claim 41, wherein manganese is administered as at least one of manganese gluconate, manganese carbonate, manganese acetate, manganese citrate, manganese oleate, manganese oxalate, manganese chloride, manganese borate, manganese nitrate, manganese phosphate, and/or manganese sulfate.

62. The regime/regimen according to claim 42, wherein manganese is administered as at least one of manganese gluconate, manganese carbonate, manganese acetate, manganese citrate, manganese oleate, manganese oxalate, manganese chloride, manganese borate, manganese nitrate, manganese phosphate, and/or manganese sulfate.

63. The regime/regimen according to claim 47, wherein manganese is administered as at least one of manganese gluconate, manganese carbonate, manganese acetate, manganese citrate, manganese oleate, manganese oxalate, manganese chloride, manganese borate, manganese nitrate, manganese phosphate, and/or manganese sulfate.

64. The regime/regimen according to claim 47, wherein said cosmetic/dermatological composition is a topically applicable solution, gel, emulsion, cream, oil, milk, makeup, serum, aerosol, vesicular dispersion, or artificial tanning preparation.

65. A topically applicable cosmetic/dermatological composition suited for influencing calcium channels in contractile fibers to relax and/or slacken skin tissue, said composition consisting of: (i) manganese, in the form of at least one organic or inorganic manganese salt, in an amount effective to influence calcium channels in contractile fibers to relax and/or slacken skin tissue; (ii) a bioaffecting amount of alverine or salt thereof and/or a chlorine-channel opener; (iii) water; and (iv) at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs and colorants.

66. The cosmetic/dermatological composition according to claim 65, said composition consisting of: (i) manganese, in the form of at least one organic or inorganic salt, in an amount effective to influence calcium channels in contractile fibers to relax and/or slacken skin tissue, said amount being from 0.001% to 0.5% by weight of the composition; (ii) a bioaffecting amount of alverine or salt thereof and/or a chlorine-channel opener; (iii) water; and (iv) at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents, dyestuffs and colorants.

67. A topically applicable cosmetic/dermatological composition suited for influencing calcium channels in contractile fibers to relax and/or slacken skin tissue, said composition consisting of: (i) at least one form of manganese selected from the group consisting of organic salts of manganese, inorganic salts of manganese, manganese-rich plant extracts and manganese-rich microorganism extracts, in an amount effective to influence calcium channels in contractile fibers to relax and/or slacken skin tissue; (ii) a bioaffecting amount of alverine or salt thereof and/or a chlorine-channel opener; (iii) water; and (iv) at least one member selected from the group consisting of topically applicable, physiologically acceptable alcohols, oils, fatty acids, waxes, emulsifiers, hydrophilic gelling agents, lipophilic gelling agents, preservatives, antioxidants, fragrances, fillers, UV-screening agents and colorants.

68. The cosmetic/dermatological composition according to claim 65, wherein the manganese is present as at least one of manganese gluconate, manganese carbonate, manganese acetate, manganese citrate, manganese oleate, manganese oxalate, manganese chloride, manganese borate, manganese nitrate, manganese phosphate, and/or manganese sulfate.

69. The cosmetic/dermatological composition according to claim 65, said composition being in the form of a topically applicable solution, gel, emulsion, cream, oil, milk, makeup, serum, aerosol, vesicular dispersion, or artificial tanning preparation.

70. The cosmetic/dermatological composition according to claim 66, wherein the manganese is present as at least one of manganese gluconate, manganese carbonate, manganese acetate, manganese citrate, manganese oleate, manganese oxalate, manganese chloride, manganese borate, manganese nitrate, manganese phosphate, and/or manganese sulfate.

71. The cosmetic/dermatological composition according to claim 66, said composition being in the form of a topically applicable solution, gel, emulsion, cream, oil, milk, makeup, serum, aerosol, vesicular dispersion, or artificial tanning preparation.

72. The cosmetic/dermatological composition according to claim 67, wherein the manganese is present as at least one of manganese gluconate, manganese carbonate, manganese acetate, manganese citrate, manganese oleate, manganese oxalate, manganese chloride, manganese borate, manganese nitrate, manganese phosphate, and/or manganese sulfate.

73. The cosmetic/dermatological composition according to claim 67, said composition being in the form of a topically applicable solution, gel, emulsion, cream, oil, milk, makeup, serum, aerosol, vesicular dispersion, or artificial tanning preparation.
